(19) United States
(12) Patent Application Publication
(10) Pub. No.: US 2013/0011354 A1
(43) Pub. Date: Jan. 10, 2013

(54) Compositions For Enhancing Hair Growth

(75) Inventors: Anuradha V. Gore, Irvine, CA (US); Kevin S. Warner, Anaheim, CA (US)

(73) Assignee: Allergan, Inc., Irvine, CA (US)

(21) Appl. No.: 13/509,040

(22) PCT Filed: Nov. 9, 2010
(86) PCT No.: PCT/US10/56036

§ 371 Date: Sep. 11, 2012

Related U.S. Application Data
(60) Provisional application No. 61/260,163, filed on Nov. 11, 2009.
(60) Provisional application No. 61/259,368, filed on Nov. 9, 2009.

(51) Int. Cl. A61K 8/42; A61Q 7/00; A61K 8/06

(52) U.S. Cl. 424/70.1; 514/613

(57) ABSTRACT

Methods and compositions for stimulating the growth of hair are disclosed wherein emulsions include a cyclopentane heptanoic acid, 2-cycloalkyl or arylalkyl compound represented by the formula I wherein the dashed bonds represent a single or double bond which can be in the cis or trans configuration, A, B, Z, X, R₁ and R₂ are as defined in the specification. Such compositions are used in treating the skin or scalp of a human or non-human animal.

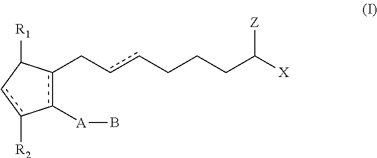

### RELATED APPLICATION

This application claims the benefit of U.S. Provisional Application Ser. No. 61/260,163, filed Nov. 11, 2009 and U.S. Provisional Application Ser. No. 61/259,368, filed Nov. 9, 2009, each disclosure of which is hereby incorporated in its entirety herein by reference.

### FIELD OF THE INVENTION

This invention relates to compositions and methods for stimulating the growth of mammalian hair comprising the application to mammalian skin of prostaglandins, prostaglandin derivatives and prostamides such as cyclopentane heptanoic acid, 2-cycloalkyl or arylalkyl derivative or a pharmacologically acceptable acid addition salt thereof, alone in association with a topical pharmaceutical carrier such as an emulsion.

### BACKGROUND OF THE INVENTION

Dermatologists recognize many different types of hair loss, the most common by far being “alopecia” wherein human males begin losing scalp hair at the temples and on the crown of the head as they get older. While this type of hair loss is largely confined to males, hence its common name “male pattern baldness,” it is not unknown in women. No known cure has yet been found despite continuing attempts to discover one.

A good deal is known about various types of human hair and its growth patterns on various parts of the body. For purposes of the present invention, it is necessary to consider various types of hair, including, terminal hairs and vellus hairs and modified terminal hairs, such as seen in eye lashes and eye brows. Terminal hairs are coarse, pigmented, long hairs in which the bulb of the hair follicle is seated deep in the dermis. Vellus hairs, on the other hand, are fine, thin, non-pigmented short hairs in which the hair bulb is located superficially in the dermis. As alopecia progresses, a transition takes place in the area of approaching baldness wherein the hairs themselves are changing from the terminal to the vellus type.

Another factor that contributes to the end result is a change in the cycle of hair growth. All hair, both human and animal, passes through a life cycle that includes three phases, namely, the anagen phase, the catagen phase and the telogen phase. The anagen phase is the period of active hair growth and, insofar as scalp hair is concerned, this generally lasts from 3-5 years. The catagen phase is a short transitional phase between the anagen and telogen phases which, in the case of scalp hair, lasts only 1-2 weeks. The final phase is the telogen phase which, for all practical purposes, can be denominated a “resting phase” where all growth ceases and the hair eventually is shed preparatory to the follicle commencing to grow a new one. Scalp hair in the telogen phase is also relatively short-lived, some 3-4 months elapsing before the hair is shed and a new one begins to grow.

Under normal hair growth conditions on the scalp, approximately 88% of the hairs are in the anagen phase, only 1% in catagen and the remainder in telogen. With the onset of male pattern baldness, a successively greater proportion of the hairs are in the telogen phase with correspondingly fewer in the active growth anagen phase.

Alopecia is associated with the severe diminution of hair follicles. A bald human subject will average only about 306 follicles per square centimeter, whereas, a non-bald human in the same age group will have an average of 460 follicles per square centimeter.

This amounts to a one-third reduction in hair follicles which, when added to the increased proportion of vellus hair follicles and the increased number of hair follicles in the telogen phase, is both significant and noticeable. Approximately 50% of the hairs must be shed to produce visible thinning of scalp hair. It is thus a combination of these factors: transition of hairs from terminal to vellus, increased number of telogen hairs—some of which have been shed, and loss of hair follicles that produces “baldness”.

While a good deal is known about the results of male pattern baldness, very little is known about its cause. The cause is generally believed to be genetic and hormonal in origin although, the known prior art attempts to control it through hormone adjustment have been singularly unsuccessful.

One known treatment for male pattern alopecia is hair transplantation. Plugs of skin containing hair are transplanted from areas of the scalp where hair is growing to bald areas with reasonable success; however, the procedure is a costly one in addition to being time-consuming and quite painful. Furthermore, the solution is inadequate from the standpoint that it becomes a practical, if not an economic, impossibility to replace but a tiny fraction of the hair present in a normal healthy head of hair.

Other non-drug related approaches to the problem include such things as ultra-violet radiation, massage, psychiatric treatment and exercise therapy. None of these, however, has been generally accepted as being effective. Even such things as revascularization surgery and acupuncture have shown little, if any, promise.

By far, the most common approach to the problem of discovering a remedy for hair loss and male pattern alopecia has been one of drug therapy. Many types of drugs ranging from vitamins to hormones have been tried and only recently has there been any indication whatsoever of even moderate success. For instance, it was felt for a long time that since an androgenic hormone was necessary for the development of male pattern baldness, that either systemic or topical application of an antiandrogenic hormone would provide the necessary inhibiting action to keep the baldness from occurring. The theory was promising but the results were uniformly disappointing.

The androgenic hormone testosterone was known, for example, to stimulate hair growth when applied topically to the deltoid area as well as when injected into the beard and pubic regions. Even oral administration was found to result in an increased hair growth in the beard and pubic areas as well as upon the trunk and extremities. While topical application to the arm causes increased hair growth, it is ineffective on the scalp and some thinning may even result. Heavy doses of testosterone have even been known to cause male pattern alopecia.

Certain therapeutic agents have been known to induce hair growth in extensive areas of the trunk, limbs and even occasionally on the face. Such hair is of intermediate status in that it is coarser than vellus but not as coarse as terminal hair. The hair is generally quite short with a length of 3 cm. being about maximum. Once the patient ceases taking the drug, the hair reverts to whatever is normal for the particular site after six months to a year has elapsed. An example of such a drug is diphenylhydantoin which is an anticonvulsant drug widely used to control epileptic seizures. Hypertrichosis is frequently observed in epileptic children some two or three months after starting the drug and first becomes noticeable on the extensor aspects of the limbs and later on the trunk and face. (The same pattern of hypertrichosis is sometimes caused by injury to the head.) As for the hair, it is often shed when the drug is discontinued but may, in some circumstances, remain.

Streptomycin is another drug that has been found to produce hypertrichosis, in much the same way as diphenylhydantoin, when administered to children suffering from tuberculous meningitis. About the same effects were observed and the onset and reversal of the hypertrichosis in relation to the period of treatment with the antibiotic leave little question but that it was the causative agent.

Two treatments have been demonstrated as showing some promise in reversing male pattern alopecia. These treatments include the use of a microemulsion cream containing both estradiol and oxandrolone as its active ingredients and the use of organic silicon.

In addition to the foregoing, it has been reported in U.S. Pat. Nos. 4,139,619 and 4,968,812 that the compound minoxidil is useful for the treatment of male pattern baldness. That compound, among others, has proven to have considerable therapeutic value in the treatment of severe hypertension. It is a so-called “vasodilator” which, as the name implies, functions to dilate the peripheral vascular system. Dermatologists and others have recognized that prolonged vasodilation of certain areas of the human body other than the scalp sometimes result in increased hair growth even in the absence of any vasodilating therapeutic agent. For instance, increased hair growth around surgical scars is not uncommon. Similarly, arteriovenous fistula have been known to result in increased vascularity accompanied by enhanced hair growth. Externally-induced vasodilation of the skin, such as, for example, by repeated biting of the limbs by the mentally retarded and localized stimulation of the shoulders by water carries has been known to bring on hypertrichosis in the affected areas. Be that as it may, similar techniques such as continued periodic massage of the scalp have been found to be totally ineffective as a means for restoring lost hair growth to the scalp. Scar tissue on the scalp inhibits rather than promotes hair growth.

U.S. Pat. No. 6,262,105 to Johnstone suggests that prostaglandins and derivatives thereof are useful in a method of enhancing hair growth.

Bimatoprost, which is sold by Allergan, Inc. of Irvine, Calif., U.S.A. as Lumigan® ophthalmic solution, for treating glaucoma now has been found as being effective to increase the growth of eyelashes when applied in the FDA approved manner.

It is, therefore, a principal object of the present invention to provide a novel and effective treatment for the stimulation of hair growth and the treatment of male pattern baldness.

Another object of the invention is to provide a method of stimulating hair growth in humans and non-human animals that is compatible with various types of therapeutic agents (e.g., Minoxidil® or Propecia®) or carriers and, therefore, would appear to be combinable with those which, by themselves, demonstrate some therapeutic activity such as, for example, microemulsion creams or topical compositions containing estradiol and oxandrolone, minoxidil or agents that block the conversion of testosterone to dihydrotesterone (Propecia®).

Still another objective is the provision of a treatment for the stimulation of hair growth which, while effective for its intended purpose, is apparently non-toxic and relatively free of unwanted side effects.

An additional object of the invention herein disclosed and claimed is to provide a method for treating hair loss in men or women which can be applied by the patient under medical supervision no more stringent than that demanded for other topically-administered therapeutic agents.

Other objects of the invention are to provide a treatment for male pattern alopecia which is safe, simple, painless, cosmetic in the sense of being invisible, easy to apply and quite inexpensive when compared with hair transplants and the like.

### SUMMARY OF THE INVENTION

This invention provides pharmaceutical compositions including emulsions (oil-in-water, microemulsions, reverse emulsions) for topical application to enhance hair growth comprising an effective amount of a cyclopentane heptanoic acid, 2-cycloalkyl or arylallyl compound represented by the formula I

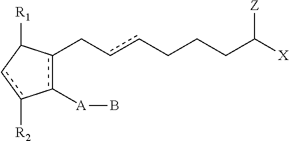

wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

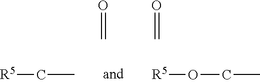

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above in free form or a pharmaceutically acceptable salt thereof, in association with a pharmaceutical carrier adapted for topical application to mammalian skin.

Preferably, the compound is a cyclopentane heptanoic acid, 2-(phenyl alkyl or

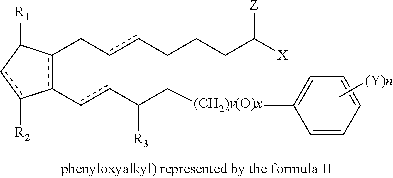

wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

More preferably the compound is a compound of formula III.

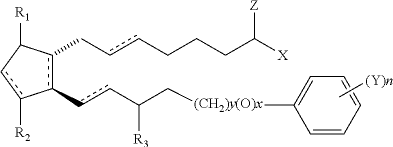

wherein hatched lines indicate α configuration, solid triangles are used to indicate β configuration.

More preferably y is 1 and x is 0 and R₁, R₂ and R₃ are hydroxy.

Most preferably the compound is cyclopentane N-ethyl heptanamide-5-cis-2-(3α-hydroxy-5-phenyl-1-trans-pentenyl)-3,5-dihydroxy, [1_{α},2_{β},3_{α},5_{α}], also known as bimatoprost.

Another aspect of the invention provides methods for stimulating the rate of hair growth and for stimulating the conversion of vellus hair or intermediate hair to growth as terminal hair in a human or non-human animal by administering to the skin of the animal an effective amount of a compound wherein the compound has the formula:

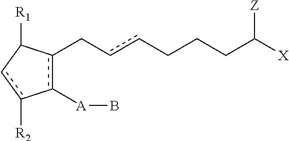

wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

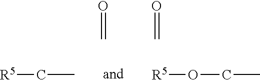

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above in free form or a pharmaceutically acceptable salt thereof.

Some embodiments of the present invention are stated below:
1. A composition for stimulating hair growth of one selected from the group consisting of eyelashes, eyebrows and scalp hair in a mammalian species comprising the application to mammalian skin of an effective amount of an emulsion of cyclopentane heptanoic acid, 2-cycloalkyl or arylalkyl compound represented by the formula I

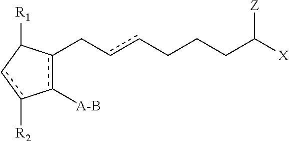

wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a eyeloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

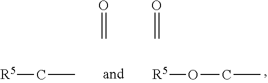

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.
2. The composition of paragraph 1 wherein the concentration of the compound applied is from about 0.0000001% to about 50% by weight of the composition.
3. The composition of paragraphs 1 and 2 wherein the compound is a compound of formula (III):

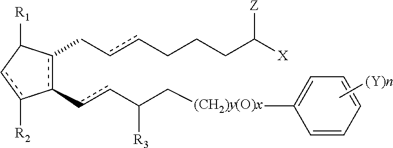

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.
4. The compositions of paragraphs 1-3 wherein the compound is bimatoprost or a pharmaceutically acceptable salt thereof.
5. The composition of paragraphs 1-4 wherein the composition is 0.005-0.03% w/w of bimatoprost.
6. The composition of paragraph 5 further comprising castor oil, polysorbate 80, glycerine, EDTA, a citric acid buffer and BAK.
7. The composition of paragraph 6 further comprising about 0.1% w/w castor oil, about 0.5% w/w polysorbate 80, about 2.2% w/w glycerine, about 0.01% w/w EDTA and 0.02% w/w BAK and other excipients selected from Tables I-IV.
8. A method for the conversion of vellus hair or intermediate hair to growth as terminal hair comprising the application to mammalian skin at the locale of vellus hair of an effective amount of an oil-in-water emulsion of cyclopentane heptanoic represented by the formula I

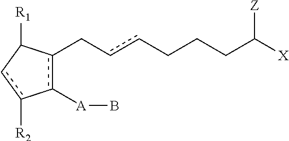

wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

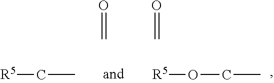

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.
9. The method of paragraph 8 wherein the concentration of the compound applied is from about 0.0000001% to about 50% by weight of the composition.
10. The method of paragraph 9 wherein the compound is a compound of the formula (III):

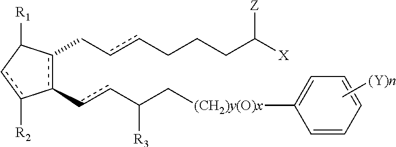

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆, hatched lines indicate α configuration and solid triangles are used to indicate β configuration.
11. The method of paragraph 10 wherein the compound applied is bimatoprost in the form of the free base or acid addition salts thereof and is present in the range of 0.01-0.03% w/v.
12. A method for stimulating hair follicles to increase hair growth of one selected from the group consisting of eyelashes, eyebrows and scalp hair and one or more properties selected from the group consisting of luster, sheen, brilliance, gloss, glow, shine or patina of hair associated with the follicles, comprising the application to mammalian skin at the locale of the follicles of an effective amount of an emulsion of cyclopentane heptanoic acid, 2-cycloalkyl or arylalkyl compound represented by the formula I

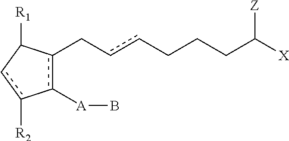

wherein the dashed bonds represent a single or double bond which can be in the cis or trans configuration, A is an alkylene or alkenylene radical having from two to six carbon atoms, which radical may be interrupted by one or more oxide radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

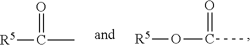

wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.
13. The method of paragraph 12 wherein the concentration of the compound applied is from about 0.0000001% to about 50% by weight of the composition.
14. The method of paragraph 13 wherein the compound is a compound of formula (III):

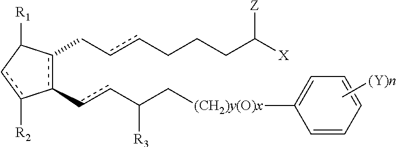

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.
15. The method of paragraph 14 wherein the compound is bimatoprost or a pharmaceutically acceptable salt and is present in the amount of 0.005-0.03% w/w.
16. The method of paragraph 15 wherein the emulsion has one or more excipients selected from Table V.
17. Use of a compound according to paragraphs 1-5 in the preparation of a medicament comprising an emulsion to increase hair growth of one selected from the group consisting of eyelashes, eyebrows and scalp hair.
18. The methods of paragraphs 1-17 further comprising administering another drug to patient to induce hair growth selected from the group consisting of Minoxidil and Propecia.

### DETAILED DESCRIPTION OF THE PREFERRED EMBODIMENT

Alopecia (baldness) a deficiency of either normal or abnormal hair, is primarily a cosmetic problem in humans. It is a deficiency of terminal hair, the broad diameter, colored hair that is readily seen. However, in the so-called bald person although there is a noticeable absence of terminal hair, the skin does contain vellus hair which is a fine colorless hair which may require microscopic examination to determine its presence. This vellus hair is a precursor to terminal hair. In accordance with the invention as described herein, compounds represented by

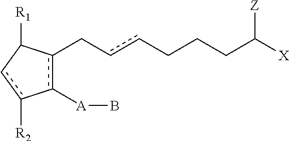

wherein R₁, R₂, A, B, Z and X are defined above, can be used to stimulate, such as stimulating the conversion of vellus hair to growth as terminal hair as well as increasing the rate of growth of terminal hair.

Some examples of representative compounds useful in the practice of the present invention include the compounds shown in Table 1:

TABLE 1
cyclopentane heptenamide-5-cis-2-(3α-hydroxy-5-phenyl-1-trans-pentenyl)-3,5-dihydroxy,
[1_{α}, 2_{β}, 3_{α}, 56_{α}]
cyclopentane N,N-dimethylheptenamide-5-cis-2-(3α-hydroxy-5-phenyl-1-trans-pentenyl)-3,
5-dihydroxy, [1_{α}, 2_{β}, 3_{α}, 5_{α}]
cyclopentane heptenylamide-5-cis-2-(3α-hydroxy-4-meta-chlorophenoxy-1-trans-pentenyl)-3,
5-dihydroxy, [1_{α}, 2_{β}, 3_{α}, 5_{α}]
cyclopentane heptenylamide-5-cis-2-(3α-hydroxy-4-trifluoromethylphenoxy-1-trans-
pentenyl)-3, 5-dihydroxy, [1_{α}, 2_{β}, 3_{α}, 5_{α}]
cyclopentane N-isopropyl heptenamide-5-cis-2-(3α-hydroxy-5-phenyl-1-trans-pentenyl)-3,
5-dihydroxy, [1_{α}, 2_{β}, 3_{α}, 5_{α}]
cyclopentane N-ethyl heptenamide-5-cis-2-(3α-hydroxy-5-phenyl-1-trans-pentenyl)-3,5
dihydroxy, [1_{α}, 2_{β}, 3_{α}, 5_{α}]
cyclopentane N-methyl heptenamide-5-cis-2-(3α-hydroxy-5-phenyl-1-trans-pentenyl)-3,
5-dihydroxy, [1_{α}, 2_{β}, 3_{α}, 5_{α}]
cyclopentane heptenamide-5-cis-2-(3α-hydroxy-4-meta-chlorophenoxy-1-trans-butenyl)-3,
5-dihydroxy, [1_{α}, 2_{β}, 3_{α}, 5_{α}]

One presently preferred compound for use in the practice of the present invention is cyclopentane N-ethyl heptanamide-5-cis-2-(3α-hydroxy-5-phenyl-1-trans-pentenyl)-3,5-dihydroxy, [1_{α},2_{β},3_{α},5_{α}], also known as bimatoprost and sold under the name of Lumigan® by Allergan, Inc., California, USA. This compound has the following structure:

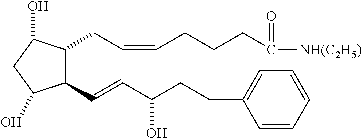

The synthesis of the above compounds described above has been disclosed in U.S. Pat. No. 5,607,978 which is hereby incorporated by reference. This patent also shows, particularly in Examples 1, 2, 5 and 7 that these compounds are not prostaglandins, in that they do not behave as prostaglandins in art-recognized assays for prostaglandin activity. The invention thus relates to the use of the above compounds, or prodrugs of the active compounds, for treatment for the stimulation of hair growth. As used herein, hair growth includes hair associated with the scalp, eyebrows, eyelids, beard, and other areas of the skin of animals.

In accordance with one aspect of the invention, the compound is mixed with a dermatologically compatible vehicle or carrier. The vehicle which may be employed for preparing compositions of this invention may comprise, for example, aqueous solutions such as e.g., physiological salines, oil solutions or ointments. The vehicle furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid; these may be used for increasing the viscosity. Furthermore, it is also possible to use soluble or insoluble drug inserts when the drug is to be administered.

The invention is also related to dermatological compositions for topical treatment for the stimulation of hair growth which comprise an effective hair growth stimulating amount of one or more compounds as defined above and a dermatologically compatible carrier. Effective amounts of the active compounds may be determined by one of ordinary skill in the art but will vary depending on the compound employed, frequency of application and desired result, and the compound will generally range from about 0.0000001 to about 50%, by weight/volume, of the dermatological composition, preferably. from about 0.001 to about 50%, w/v, of total dermatological composition, more preferably from about 0.01-0.10% w/v, or 0.1 to about 1.0% w/v or 0.01-0.03% w/v.

TABLE I
BIMATOPROST EMULSIONS I

| Ingredient | | A | B | C | D | Function |
|---|---|---|---|---|---|---|
| Bimatoprost | 0.03 | 0.01- | 0.01- | 0.01- | 0.01- | Active Ingredient |
| | | 0.03 | 0.03 | 0.03 | 0.03 | |
| Castor Oil | — | 0.25 | 0.1 | 0.1 | 0.1 | Lipophilic |
| | | | | | | Vehicle |
| Polysorbate-80 | — | 0.5 | — | — | — | Primary |
| | | | | | | Emulsifier |
| Polysorbate-20 | — | — | 0.25 | — | 0.25 | Primary |
| | | | | | | Emulsifier |
| Pemulen TR-1 | — | — | — | 0.1 | | Primary |
| | | | | | | Emulsifier |
| Pemulen TR-2 | — | 0.1 | 0.1 | — | — | Secondary |
| | | | | | | Emulsifier |
| Glycerin | | 2.2 | 2.2 | 2.2 | 2.2 | Demulcent and |
| | | | | | | Tonicity Agent |
| Citric acid, H₂O | 0.014 | — | — | — | — | Buffering Agent |
| Dibasic sodium | 0.268 | — | — | — | — | Buffering Agent |
| phosphate, 7H₂O | | | | | | |
| Benzalkonium | 0.005 | 0.01 | 0.01 | 0.01 | 0.01 | Antimicrobial |
| chloride | | | | | | Preservative |
| Sodium chloride | 0.83 | — | | | | Tonicity Agent |
| Disodium Edetate | — | 0.01 | 0.01 | 0.01 | 0.01 | Chelating agent |
| Acrylate | — | — | — | — | 0.02 | Viscosity agent |
| crosspolymer | | | | | | |
| Sodium Hydrox- | pH | pH | pH | pH | pH | pH adjustment |
| ide | 7.4 | 7.4 | 7.4 | 7.4 | 7.4 | |
| Purified Water | QS | QS | QS | QS | QS | Hydrophilic |
| | | | | | | Vehicle |

TABLE II
BIMATOPROST EMULSIONS II

| | Proposed Emulsion Formulations | | | | |
|---|---|---|---|---|---|
| Ingredient | E | F | G | H | Function |
| Bimatoprost | 0.01-0.03 | 0.01-0.03 | 0.01-0.03 | 0.01-0.03 | Active |
| | | | | | Ingredient |
| Castor Oil | 0.1 | — | 0.1 | 0.25 | Lipophilic |
| | | | | | Vehicle |
| Squalane | — | 0.1 | — | — | Lipophilic |
| | | | | | Vehicle |
| Polysor- | — | 0.25 | — | 0.25 | Primary |
| bate-20 | | | | | Emulsifier |
| POE-40- | 0.25 | — | 0.25 | — | Primary |
| Stearate | | | | | Emulsifier |
| Solutol- | — | — | — | 0.25 | Primary |
| 15-HS | | | | | Emulsifier |
| Pemulen | | | 0.1 | — | Primary |
| TR-1 | | | | | Emulsifier |
| Pemulen | 0.1 | 0.1 | — | — | Secondary |
| TR-2 | | | | | Emulsifier |
| POE-40- | — | — | — | — | Secondary |
| Stearate | | | | | Emulsifier |
| Glycerin | 2.2 | 2.2 | 2.2 | 1.2 | Demulcent |
| | | | | | and Tonicity |
| | | | | | Agent |
| Boric | — | — | — | 0.6 | Buffering |
| Acid | | | | | Agent |
| Benzal- | 0.01 | 0.01 | 0.01 | — | Antimicrobial |
| konium | | | | | Preservative |
| chloride | | | | | |
| Disodium | 0.01 | 0.01 | 0.01 | — | Chelating |
| Edetate | | | | | agent |
| Acrylater | — | 0.02 | 0.02 | — | Viscosity |
| crosspolymer | | | | | agent |
| HPMC | 2.0 | — | — | 2.0 | Viscosity |
| | | | | | agent |
| Purite | — | — | — | 0.01 | Antimicrobial |
| | | | | | Preservative |
| Sodium | pH 7.4 | pH 7.4 | pH 7.4 | pH 7.4 | pH |
| Hydroxide | | | | | adjustment |
| Purified | QS | QS | QS | QS | Hydrophilic |
| Water | | | | | Vehicle |

The present invention finds application in all mammalian species, including both humans and animals. In humans, the compounds of the subject invention can be applied for example, to the scalp, face, beard, head, pubic area, upper lip, eyebrows, and eyelids. In animals raised for their pelts, e.g., mink, the compounds can be applied over the entire surface of the body to improve the overall pelt for commercial reasons. The process can also be used for cosmetic reasons in animals, e.g., applied to the skin of dogs and cats having bald patches due to mange or other diseases causing a degree of alopecia.

The pharmaceutical compositions contemplated by this invention include pharmaceutical emulsions suited for topical and local action. The term “topical” as employed herein relates to the use of a compound, as described herein, incorporated in a suitable pharmaceutical carrier with an optional emulsifier, and applied at the site of thinning hair or baldness for exertion of local action. Accordingly, such topical compositions include those pharmaceutical forms in which the compound is applied externally by direct contact with the skin surface to be treated. Conventional pharmaceutical forms for this purpose include emulsions, dispersions, reverse emulsions, micro emulsions, ointments, liniments, creams, shampoos, lotions, pastes, jellies, sprays, aerosols, and the like, and may be applied in patches or impregnated dressings depending on the part of the body to be treated. The term “ointment” embraces formulations (including creams) having oleaginous, water-soluble and emulsion-type bases, e.g., petrolatum, lanolin, polyethylene glycols, as well as mixtures of these.

Typically, the compounds are applied repeatedly for a sustained period of time topically on the part of the body to be treated, for example, the eyelids, eyebrows, skin or scalp. The preferred dosage regimen will generally involve regular, such as daily, administration for a period of treatment of at least one month, more preferably at least three months, and most preferably at least six months.

For topical use on the eyelids or eyebrows, the active compounds can be formulated in emulsions, aqueous solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, emulsions and aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and compound to be used, various amounts of the drug and different dose regimens may be employed. Typically, the daily amount of compound for treatment of the eyelid may be about 0.1 ng to about 100 mg per eyelid.

For topical use on the skin and the scalp, the compound can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like.

Various matrices for slow release delivery may also be used. Typically, the dose to be applied on the scalp is in the range of about 0.1 ng to about 100 mg per day, more preferably about 1 ng to about 10 mg per day, and most preferably about 10 ng to about 1 mg per day depending on the compound and the formulation. To achieve the daily amount of medication depending on the formulation, the compound may be administered once or several times daily with or without antioxidants.

Skin penetration data was gathered on the following formulations.

TABLE III
Formulations

| | 0.03% | 0.03% | 0.015% | 0.015% | |
|---|---|---|---|---|---|
| | PT4 | PT8 | PT4 | PT8 | Latisse |
| Ingredient | % w/w | % w/w | % w/w | % w/w | % w/w |
| Castor oil | 0.100 | 0.100 | 0.100 | 0.100 | — |
| Polysorbate 80 | 0.500 | 0.500 | 0.500 | 0.500 | — |
| Glycerin | 2.200 | 2.200 | 2.200 | 2.200 | — |
| EDTA | 0.010 | 0.010 | 0.010 | 0.010 | — |
| BAK | 0.015 | 0.015 | 0.015 | 0.015 | 0.005 |
| Bimatoprost | 0.030 | 0.030 | 0.015 | 0.015 | 0.030 |
| Sodium | 0.268 | 0.268 | 0.268 | 0.268 | 0.268 |
| phosphate | | | | | |
| dibasic | | | | | |
| heptahydrate | | | | | |
| Citric acid | 0.014 | 0.014 | 0.014 | 0.014 | 0.014 |
| monohydrate | | | | | |
| sodium | — | — | — | — | 0.830 |
| chloride | | | | | |
| HPMC | 0.250 | 0.500 | 0.250 | 0.500 | — |
| Water | qs | qs | qs | qs | qs |
| Viscosity (cPs) | 4 | 24 | 4 | 24 | ~1 |

The following protocol was followed:

| | |
|---|---|
| Test Article | AGN-192024 Formulations |
| | 0.003% PT4 |
| | 0.015% PT8 |
| | 0.015% PT4 |
| | 0.015% PT8 |
| | 0.03% Solution (LUMIGAN) |
| Test System | Ex vivo human female skin |
| | eyelid (n = 3 donors, 2 replicates/donor)— |
| | inner tissues removed |
| | Nominal 1.0 cm² Franz Diffusion Cells |
| Dose | 5 μL/cm²/skin section |
| PK | Reservoir solution at 0, 2, 4, 12, and 24 hours |
| Sampling | Stratum corneum, epidermis and dermis at 24 hours |
| | (surface wash, glass rod) |
| | SC strip tape: first 1-2 separated from 3-10 |
| | Surface wash: Water/Ethanol = 75/25 |

### Results:

The preliminary results indicate that eye lid skin penetration of bimatoprost is not statistically different between 0.03% PT4; 0.03% PT8; and Latisse solution. The bimatoprost penetration for the 0.015% formulations are approximately 50-70% of the Latisse control. Further, the rank order for bimatoprost concentration in the epidermis, and dermis tissues is Latisse >0.03% PT4>0.03% PT8. The tissue concentration results for the 0.015% bimatoprost formulations show a similar trend.

### Aesthetics:

Viscosity measurements (see composition table above for values) for each formulation confirm the results of the aesthetics evaluation. PT 4 and PT 8 vehicles feel smooth on the skin. Neither formulations leaves a residue upon evaporation (a concern because of the thickener HPMC in the formulations) and both dried cleanly on the skin. No observed differences were noted when dispensing PT 4 and PT 8 vehicles and Lumigan placebo from the bottle.

Other excipients for use in bimatoprost emulsions of the present invention include:

TABLE IV
ADDITIONAL EMULSION EXCIPIENTS

| | | | | expected |
|---|---|---|---|---|
| | | Chemical | CAS | conc. range |
| Category | ingredient | name | number | % w/v |
| Active ingredient | Bimatoprost | | | 0.001-0.03 |
| Oil phase | Castor oil | Castor oil | 8001-79-4 | 0.1-0.5 |
| | Olive oil | Olive oil | 8001-25-0 | 0.1-0.25 |
| | Squalane | 2,6,10,15,19,23- | 111-01-3 | 0.1-0.5 |
| | | Hexamethyl- | | |
| | | tetracosane | | |
| | Caprylic/capric | Medium chain | 65381-09-1 | 0.1-0.5 |
| | triglycerides | triglycerides | | |
| Surfactant | Polysorbate 80 | Polyoxyethylene (20) | 9005-65-6 | 0.25-1.0 |
| (primary emulsifier) | | sorbitan | | |
| | | monooleate | | |
| | Pemulen TR-1 | Carbomer 1342 | | 0.05-0.2 |
| | | Acrylates/C10-30 | | |
| | | Alkyl acrylate | | |
| | | crosspolymer | | |
| | Polysorbate 20 | Polyoxyethylene | 9005-64-5 | 0.25-1.0 |
| | | sorbitan | | |
| | | monolaurate | | |
| Co surfactant | Pemulen TR-2 | Carbomer 1342 | | 0.05-0.2 |
| | | Acrylates/C10-30 | | |
| | | Alkyl acrylate | | |
| | | crosspolymer | | |
| | Solutol | polyethyl- | | 0.1-0.5 |
| | | eneglycol 660 | | |
| | | hydroxystearate | | |
| | POE-40-Stearate | Polyoxyethylene (40) | | 0.1-0.5 |
| | | Stearate Myrj52 | | |
| Tonicity/emollient | Glycerin | Glycerol | 8043-29-6 | 2-2.6 |
| Viscosity agent | Carbopol ETD | Acrylates/C10-30 | | 0.02-0.1 |
| | 2020 | Alkyl acrylate | | |
| | | crosspolymer | | |
| | Ultrez 10 | Carbomer | | 0.02-0.1 |
| | | interpolymer type A | | |
| | | Acrylates/C10-30 | | |
| | | Alkyl acrylate | | |
| | | crosspolymer | | |
| | Hydroxypropyl | | 9004-67-3 | 0.5-2 |
| | Methyl | | | |
| | cellulose | | | |
| | Povidone | Povidone (K30, K90) | 9003-39-8 | 0.5-2 |
| | Methyl cellulose | | 9004-67-5 | 0.5-2 |
| | Hyaluronic acid | hyaluronate | | 0.5 |
| | (high MW) | | | |
| | Carboxymethyl- | low, medium | | 0.1-1.0 |
| | cellulose | or high viscosity | | |
| | | polymer | | |
| Neutralizing | Sodium Hydroxide | | | qs for pH~7.4 |
| Agents (pH | Triethanolamine | iris-(2- | 102-71-6 | qs for pH~7.4 |
| adjustment) | | Hydroxyethyl) | | |
| | | amine | | |
| Buffer | Phosphate/citrate | | | 0.01 |
| | Boric acid | | | |
| Preservative | Benzalkonium | | 8001-54-5 | 0.002-0.02 |
| | chloride | | | |
| | Purite | | | 0.005-0.012 |
| | Methylparaben | | 99-76-3 | 0.01-0.05 |
| | Propylparaben | | 94-13-3 | 0.01 |
| Chelating agent | EDTA | Ethylenediamine | 6381-92-6 | 0.005-0.01 |
| | | tetraacetic acid | | |

The invention is further illustrated by the following non-limiting examples:

TABLE V
Further Formulations

| | Latisse ® | Formulation 1 | Formulation 2 |
|---|---|---|---|
| Ingredient | % w/w | % w/w | % w/w |
| Bimatoprost | 0.03 | (0.005-0.03%) | (0.005-0.03%) |
| Castor oil | — | 0.1 | 0.1 |
| Polysorbate 80 | — | 0.5 | 0.5 |
| Glycerin | — | 2.2 | 2.2 |
| EDTA | — | 0.01 | 0.01 |
| BAK | 0.005 | 0.015 | 0.015 |
| HPMC polymer | — | 0.25 | 0.5 |
| HEC polymer | — | — | — |
| Sodium phosphate | 0.268 | 0.268 | 0.268 |
| dibasic | | | |
| heptahydrate | | | |
| Citric acid | 0.014 | 0.014 | 0.014 |
| monohydrate | | | |
| Sodium chloride | 0.82 | — | — |
| Water | qs | qs | qs |
| pH | 7.3 | 7.3 | 7.3 |
| Viscosity range | Water | Light | Medium |
| | (~1 cps) | (4-8 cps) | (10-50 cps) |

### Example 1

### In Vivo Treatment Eyelash growth

A study is initiated to systematically evaluate the length and appearance of eyelashes of patients who are administering a bimatoprost emulsion on only one eyelid. The study involves 10 subjects, 5 male, 5 female ranging from 22-38 years). Each subject is treated daily by the topical application of one drop of bimatoprost emulsion at a dosage of 1.5 .mu.g/ml/eyelid/day (0.01% why ophthalmic emulsion, Formulation 1, Table V) to the region of the eyelid by instilling the drop onto the surface of the eyelid. The other eyelid is not treated with bimatoprost emulsion and served as a control. Observations are made under high magnification at the slit lamp biomicroscope. Documentation of differences between the control and treatment areas is accomplished using a camera specially adapted for use with the slit lamp biomicroscope.

The results of the observations are as follows:

Length of lashes: Increased length of eyelashes will be observed on the side treated with bimatoprost emulsion. The difference in length is expected to vary from approximately 10% to as much as 30%.

Number of lashes: An increased numbers of lashes will be observed in the treated eye of each patient. In areas where there are a large number of lashes in the control eye, the increased number of lashes in the bimatoprost-treated eye gave the lashes on the treated side a more thickly matted overall appearance.

In areas where there are lash-like hairs above the lash line and in the medial and lateral canthal areas, the hairs will be much longer, thicker and heavier. They also leave the surface of the skin at a more acute angle, as though they are stiffer or held in a more erect position by more robust follicles. This greater incline, pitch, rise or perpendicular angulation from the skin surface gives the appearance of greater density of the hairs.

The foregoing observations clearly establish that 0.01% bimatoprost emulsions as described herein can be used to increase the growth of hair in man. This conclusion is based on the regular and consistent finding of manifestations of increased hair growth in treated vs. control areas in human subjects. The conclusion that the bimatoprost emulsion is capable of inducing increased robust growth of hair is based not on a single parameter, i.e., length, but is based on multiple lines of evidence as described in the results. Detailed examination and description of multiple parameters of differences in hair is greatly facilitated by the ability to examine the hairs at high magnification under stable conditions of fixed focal length and subject position utilizing the capabilities of the slitlamp biomicroscope.

### Example 2

### In Vivo Treatment Alopecia

A study is initiated to systematically evaluate the appearance of thinning hair on the scalp of patients who are administering a bimatoprost emulsion on their scalps for the treatment of alopecia. The study involves 10 subjects, 5 male, 5 female, average age 60 years, (ranging from 40-84 years). Each subject is treated daily by the topical application of one drop of bimatoprost emulsion at a dosage of 1.5.mu.g/ml/eye/day (0.03% w/w bimatoprost emulsion, Formula II, Table V) to the region of the scalp experiencing hair loss by instilling three drops of emulsion onto the surface of the scalp twice a day.

Observations are made under high magnification at the slit lamp biomicroscope. Documentation of differences between the control and treatment areas is accomplished using a camera specially adapted for use with the slit lamp biomicroscope.

The results of the observations are as follows:

Length of hair: Increased length of hair will be observed on the side treated with bimatoprost emulsion. The difference in length varies from approximately 7% to as much as 32%.
Number of hairs: Increased hair growth will be observed in the treated areas of each patient.

The foregoing observations clearly establish that 0.03% bimatoprost emulsions can be used to increase the growth of hair in humans. This conclusion is based on the regular and consistent finding of manifestations of increased hair growth in treated vs. control areas in human subjects.

### In Vivo Treatment Alopecia

### Example 3

A study is initiated to systematically evaluate the appearance of thinning eyebrows of patients who are administering a 0.015% w/w bimatoprost emulsion on their eyebrows for the treatment of alopecia. The study involves 10 subjects, 5 male, 5 female, average age 60 years, (ranging from 40-84 years). Each subject is treated daily by the topical application of one drop of bimatoprost emulsion at a dosage of 1.5.mu.g/ml/eye/day (0.015%, by weight/volume, ophthalmic emulsion) to the region of the eyebrows by instilling one drop onto the surface of the scalp.

The study is limited to subjects who have administered bimatoprost to one eyebrow for more than 3 months. The mean duration of exposure to bimatoprost prior to assessing the parameter of hair growth on the eyebrow between the control and study eye is 129 days (range 90-254 days). Observations are made under high magnification at the slit lamp biomicroscope. Documentation of differences between the control and treatment areas is accomplished using a camera specially adapted for use with the slit lamp biomicroscope.

The results of the observations are as follows:

Length of eyebrows: Increased length of eyebrows will be regularly observed on the side treated with bimatoprost emulsion. The difference in length varies from approximately 10% to as much as 30%.
Number of hairs: Increased numbers of eyebrows will be observed in the treated areas of each patient. In areas where there are a large number of hairs in the control are, the increased numbers of hair in the bimatoprost-emulsion area gave the hair on the treated side a more thickly matted overall appearance.

The foregoing observations clearly establish that 0.015% bimatoprost emulsions can be used to increase the growth of eyebrows in humans. This conclusion is based on the regular and consistent finding of manifestations of increased hair growth in treated vs. control areas in human subjects. The conclusion that the drug bimatoprost is capable of inducing increased robust growth of hair is based not on a single parameter, i.e., length, but is based on multiple lines of evidence as described in the results. Detailed examination and description of multiple parameters of differences in hair is greatly facilitated by the ability to examine the hairs at high magnification under stable conditions of fixed focal length and subject position utilizing the capabilities of the slitlamp biomicroscope.

1. A composition for stimulating hair growth of one selected from the group consisting of eyelashes, eyebrows and scalp hair in a mammalian species comprising the application to mammalian skin of an effective amount of an emulsion of cyclopentane heptanoic acid, 2-cycloalkyl or arylalkyl compound represented by the formula I 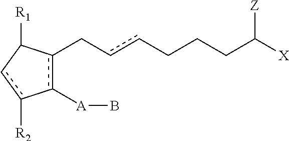
wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 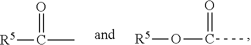
wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

2. The composition of claim 1 wherein the concentration of the compound applied is from about 0.0000001% to about 50% by weight of the composition.

3. The composition of claim 2 wherein the compound is a compound of formula (III). 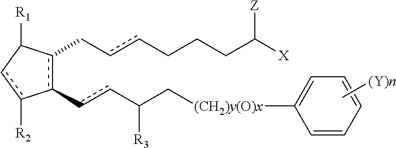
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

4. The compositions of claim 3 wherein the compound is bimatoprost or a pharmaceutically acceptable salt thereof.

5. The composition of claim 4 wherein the composition is 0.005-0.03% w/w of bimatoprost.

6. The composition of claim 5 further comprising castor oil, polysorbate 80, glycerine, EDTA, a citric acid buffer and BAK.

7. The composition of claim 6 further comprising about 0.1% w/w castor oil, about 0.5% w/w polysorbate 80, about 2.2% w/w glycerine, about 0.01% w/w EDTA and 0.02% w/w BAK.

8. A method for the conversion of vellus hair or intermediate hair to growth as terminal hair comprising the application to mammalian skin at the locale of vellus hair of an effective amount of an oil-in-water emulsion of cyclopentane heptanoic represented by the formula I 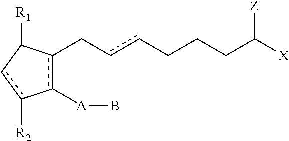
wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 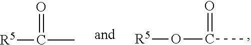
wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

9. The method of claim 8 wherein the concentration of the compound applied is from about 0.0000001% to about 50% by weight of the composition.

10. The method of claim 9 wherein the compound is a compound of the formula (III): 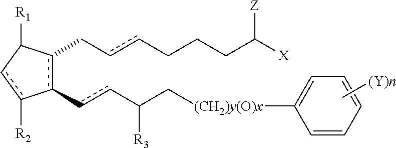
wherein y is 0 or 1, x is 0 or 1 and x and y arc not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆, hatched lines indicate α configuration and solid triangles are used to indicate β configuration.

11. The method of claim 10 wherein the compound applied is bimatoprost in the form of the free base or acid addition salts thereof and is present in the range of 0.01-0.03% w/v.

12. A method for stimulating hair follicles to increase hair growth of one selected from the group consisting of eyelashes, eyebrows and scalp hair and one or more properties selected from the group consisting of luster, sheen, brilliance, gloss, glow, shine or patina of hair associated with the follicles, comprising the application to mammalian skin at the locale of the follicles of an effective amount
of an emulsion of cyclopentane heptanoic acid, 2-cycloalkyl or arylalkyl compound represented by the formula I 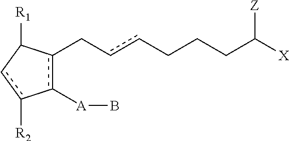
wherein the dashed bonds represent a single or double bond which can be in the cis or trans configuration, A is an alkylene or alkenylene radical having from two to six carbon atoms, which radical may be interrupted by one or more oxide radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 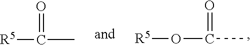
wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

13. The method of claim 12 wherein the concentration of the compound applied is from about 0.0000001% to about 50% by weight of the composition.

14. The method of claim 13 wherein the compound is a compound of formula (III): 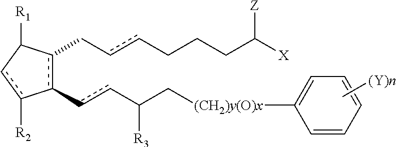
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

15. The method of claim 14 wherein the compound is bimatoprost or a pharmaceutically acceptable salt and is present in the amount of 0.005-0.03% w/w.

16. The method of claim 15 wherein the emulsion has one or more excipients selected from Table V.
